(19) United States
(12) Patent Application Publication
(10) Pub. No.: US 2005/0131305 A1
(43) Pub. Date: Jun. 16, 2005

(54) Sensor unit and method for sensing a blood related parameter and system including such a sensor unit

(76) Inventors: Bo G Danielson, Uppsala (SE); Dick Persson, Los (SE)

Correspondence Address: JACOBSON HOLMAN PLLC, 400 SEVENTH STREET N.W., SUITE 600, WASHINGTON, DC, 20004, US

(21) Appl. No.: 10/514,635

(22) PCT Filed: May 14, 2003
(86) PCT No.: PCT/SE03/00784

(30) Foreign Application Priority Data
May 17, 2002 (SE) 02014975

(51) Int. Cl. A61B 5/00; A61B 5/02; A61M 31/00

(52) U.S. Cl. 600/481; 600/365; 600/309; 600/486; 604/503

(57) ABSTRACT

A sensor unit (5) and a method for sensing a blood related parameter in a bloodstream of a human or an animal, including sensing the blood related parameter(s) in a channel (7) of a lid member for a main body of a bloodstream access device (1) which channel allows a continuous blood flow passing between an artery side and a vein side of the bloodstream access device (1). The invention also concerns a method for monitoring the function of a bloodstream access device and a blood parameter sensor system.

### FIELD OF THE INVENTION

The invention concerns a sensor unit for sensing a blood related parameter in a bloodstream of a human or an animal. It also concerns a method for monitoring the function of a bloodstream access device and a blood parameter sensor system including such a sensor unit.

### DESCRIPTION OF PRIOR ART

Various devices are previously known for sensing various parameters in the bloodstream of a human. The most common arrangement for measuring such parameters is by introducing a catheter including a sensor element into body tissue of the patient. This procedure however is somewhat problematic, since the sensor element may be positioned outside an intended blood vessel which can result in erroneous measurements. Another drawback is that it is often necessary to punch the patient's skin at regular intervals in order to gain proper access to the patient's bloodstream, since this solution is not suitable for permanent use.

U.S. Pat. No. 5,120,313 discloses a method for measuring blood pressure in an animal or human using a percutaneous access port. This access port includes a base assembly which is intended to be implanted into the animal or human and comprises an inlet port which is adapted to be connected to a selected organ in order to provide communication with a fluid chamber inside the base assembly. A transducer inside the fluid chamber of the base assembly is adapted to detect the pressure of the fluid so as to provide a signal indicative of the blood pressure of the human or the animal.

The described arrangement is equipped with a pump for continuously flushing the system including a catheter for connection to the bloodstream of the human or the animal in order to prevent formation of blood clots. Altogether the described arrangement is an insecure, complicated, limited and expensive solution which appears to be mainly intended for use in animals in connection with medical research.

### AIM AND MOST IMPORTANT FEATURES OF THE INVENTION

It is an aim of this invention to provide a solution to the prior art problems and in particular to provide a secure, inexpensive and easy handled sensor arrangement.

This aim is achieved in a sensor unit as above according to the features of the characterising portion of claim 1.

The inventive sensor unit provides for easy application in or on a bloodstream access device and guarantees that the sensor element is operative with respect of a continuous blood flow, thus guaranteeing that sensing of the blood related parameter is carried out accurately.

It is an advantage to use a bloodstream access device which preferably is implanted for permanent use. Normally such devices are implanted so as to allow blood treatment such as blood filtering in case of patients with a renal failure diagnosis. According to this aspect of the invention such a device is used as part of the sensor system besides its normal use for blood treatment.

One example of a preferred bloodstream access device is previously known from WO 99/20338 (Hemapure AB). It is preferred that a lid member being included in the sensor unit of the present invention is adapted to be applied to a bloodstream access device according to that document in a manner which is thoroughly described therein.

Allowing a continuous blood flow during operation of the sensor element avoids formation of clots etc. and contributes to the sensor unit according to the invention being a long life solution.

An inventive method provides for monitoring the function of the bloodstream access device. Hereby the blood temperature is sensed in a channel which allows a continuous blood flow passing between an artery side and a vein side of the bloodstream access device. The sensed value is used as an indication of the blood flow through the bloodstream access device. Low flow and low temperature might indicate malfunction of the access device for various reasons and should at least trigger a control of the device by skilled personnel.

Further advantages are achieved with respect to further aspects of the invention and will be explained in the following detailed description.

### BRIEF DESCRIPTION OF DRAWINGS

The invention will now be described in more detail at the background of preferred embodiments and with reference to the drawings, wherein:

FIG. 1a shows a bloodstream access device for use in connection with a system according to the invention,

FIGS. 2a and 2b show an arrangement including a bloodstream access device with attached sensor unit according to the invention,

FIGS. 3a and 3b show an alternative sensor unit, and

FIG. 4 shows a system including an inventive sensor unit in respect of a human.

### DESCRIPTION OF EMBODIMENTS

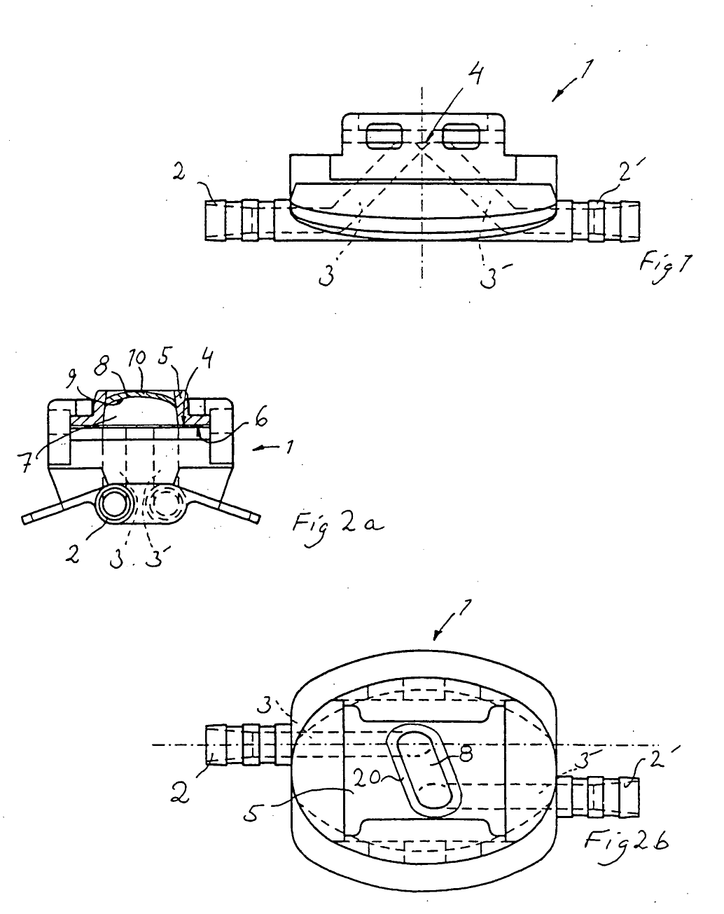

In FIG. 1 a bloodstream access device 1 is shown having nipples 2 and 2′ for connection to an artery and a vein of a human or an animal. Channels 3 and 3′ interconnect the nipples with a flat or curved interface surface 4 which is intended for connection to a connection device belonging to an external circuit. Such a circuit could include an artificial kidney. The interface is co-operating with a lid between blood treatments, said lid including a channel for allowing a continuous flow between the artery and the vein side through the channels 3 and 3′.

Fixing the different lids and connection devices onto the bloodstream access device is not part of the invention, but for understanding reference is made to the above mentioned document WO 99/20338. The device 1 is intended for permanent implantation into a human or an animal and includes means for allowing ingrowth of body tissue for stabilising purposes.

In FIG. 2a the bloodstream access device 1 in FIG. 1 is shown in a view from the right in FIG. 1 carrying a sensor unit 5 which is shown in a section. The sensor unit 5 co-operates with the bloodstream access device to that extent that it provides sealing co-operation between a sealing surface 6 and the surface of the interface 4 of the bloodstream access device 1. The sensor unit 5 is thus positioned and fixed on the bloodstream access device in a manner which is described in the above-mentioned WO document.

Further, the sensor unit 5 has a channel portion 7 which is intended to interconnect the channels 3 and 3′ of the bloodstream access device 1 so as to allow a continuous blood flow through the device with the applied sensor unit 5. At the top the channel 7 is limited by a wall including a sensor element 8 comprising a sensor layer 9 which comprises the inner wall portion of that part of the channel portion 7.

Further, the sensor element 8 includes a translucent protective layer 10 which protects the sensor layer 9 from being affected from outside mechanical influences as well as, in certain applications, provides a certain thermal insulation so as not to cool down the blood streaming through the channel 7.

The sensor layer 9 includes preferably liquid crystals which are sensitive to the parameter which at present is intended to be sensed. It may thus for example be temperature sensitive or sensitive to a defined substance in the bloodstream such as insulin or glucose. As is per se known, the sensor layer may be of a kind which changes colour so as provide a visual indication of the parameter level, be it temperature, concentration etc., of the blood being present inside the channel 7.

Temperature indication may in turn be used as a measurement of the flow through the channel 7. I.e. in principle, high flow results in high temperature; low flow results in lower temperature. Flow indication in turn may be used for monitoring the function of the bloodstream access device and absence of clots etc. However, temperature indication could also be used for monitoring the certain ill-health conditions of the patient. An ideal temperature might be for example 35° C. and higher or lower temperatures might indicate some kind of problem.

Taken more generally, the indication could be used as a warning or as a trigger for introducing medication into the patient.

In case the sensor layer includes sensor elements that are indicative of the concentration of a specific substance in the bloodstream, this may be indicated similarly, that is with liquid crystals which have the capability to change colour or in any other per se known manner.

FIG. 2b shows the bloodstream access device 1 from above with a fastened sensor unit 5. The sensor element 8 is shown as an oblong element in the centre of the sensor unit 5. In case of liquid crystal material having the provision to change colour as a response to sensed parameter values, it is preferred that the sensor element 8 is surrounded by a surface 20 having a colour scale. Hereby easy comparison with the present colour of the sensor element 8 is enabled. This makes it possible to have a quick indication on the temperature and thus the function of the device 1 or, at occasions, the concentration level etc. prevailing inside channel 7.

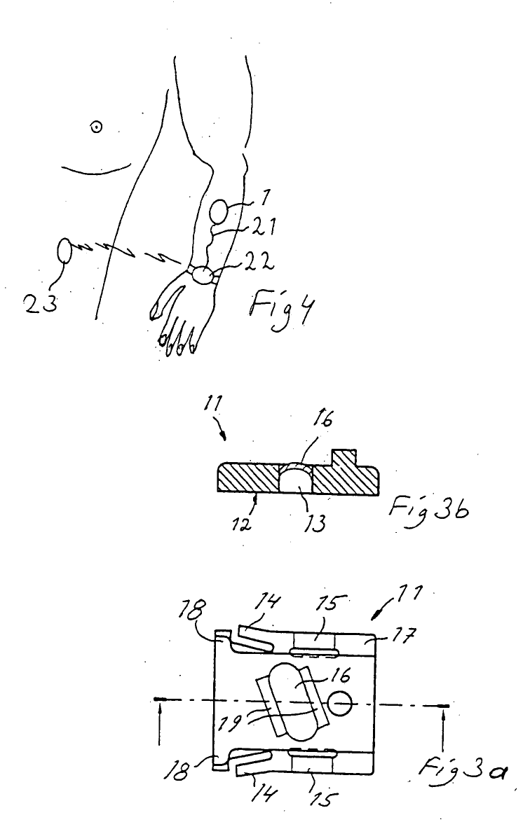

A second embodiment of a sensor unit 11 is shown in FIG. 3a. In accordance with connecting parts described in the above-mentioned WO document, the sensor unit is provided with snap wings 14, and pressing portions 15 on holding means 17. Further, locking elements or shoulders 18 are used for positioning the sensor unit 11 on a bloodstream access device 1 (FIG. 1).

An oblong sensor element 16 is positioned centrally and with an acute angle with respect to a longitudinal axis of the sensor unit 11. At the sides of the sensor element 16, there are indicated two areas 19 which may be coloured in scales so as to provide easy indication of flow, temperature, concentration etc. etc.

FIG. 3b shows the sensor unit 11 in a longitudinal section wherein the curved sensor element 16 contributes to limit a channel 13, which corresponds to the channel 7 of the sensor unit 5 in FIG. 2a.

The invention may be modified further and for example the sensor units 5 and 11, which in practice are lid members for co-operation with the bloodstream access device, may be connected to outside equipment for indicating sensed temperature, flow concentration etc. This is indicated in FIG. 4, which shows a system including an inventive sensor unit in respect of a human. In this case the lid member may be connected, for example over an electric wire 21 or wireless, with a wrist carried indicator 22 resembling a wrist-watch.

It is also possible to combine the sensor unit with other kinds of equipment. For example in case of the sensor unit includes a sensor element for sensing blood glucose level, an inventive sensor unit may be connected to an insulin pump 23 for introducing insulin into the body of the patient. A simple external device may be a warning indicator such as a buzzer, a beeper or a lamp, indicating malfunction or parameter levels below a predetermined value.

As examples, blood temperature, blood flow, blood-pressure, level of biochemical substances such as glucose, insulin, urea may be subject to being sensed by adapted sensor elements of a sensor unit according to the invention.

Further modifications may include other per se known sensor elements; use in connection with other types of bloodstream access devices; and connections to other kinds of indicators and external equipment.

1. Sensor unit including at least one sensor element for sensing a blood related parameter in a bloodstream of a human or an animal, characterized in
that the sensor unit includes a lid member for a main body of a bloodstream access device which includes connecting elements for connection to an artery and a vein of the human or the animal,
that the sensor unit comprises a channel for allowing a continuous blood flow passing between an artery side and a vein side of the bloodstream access device, and
that the sensor element(s) is (are) positioned for sensing the blood related parameter(s) in the channel.

2. Sensor unit according to claim 1, characterized in that it includes a parameter level indicator.

3. Sensor unit according to claim 1, characterized in that it includes means for transmitting parameter related signals to an external receiver.

4. Sensor unit according to claim 1, characterized in that the sensor element is a liquid crystal sensor.

5. Sensor unit according to claim 1, characterized in that the sensor element is a sensor for one parameter in the group: blood temperature, blood flow, blood-pressure, level of biochemical substances such as glucose, insulin, urea.

6. Method for monitoring the function of a bloodstream access device, characterized by
sensing blood temperature in a channel of a lid member for a main body of a bloodstream access device which channel allows a continuous blood flow passing between an artery side and a vein side of the bloodstream access device, and
using the sensed value as an indication of the blood flow through the bloodstream access device.

7. Blood parameter sensor system, characterized in that it includes a bloodstream access device and a sensor unit according to claim 1.

8. System according to claim 7, characterized in that it includes a feed-back device for administration of a substance into the human or the animal as a response to a sensed parameter level.

9. System according to claim 8, characterized in that the feed-back device includes a controlled pump for the substance.
